(19) United States
(12) Patent Application Publication
(10) Pub. No.: US 2019/0290545 A1
(43) Pub. Date: Sep. 26, 2019

(54) SOLID PHARMACEUTICAL PREPARATION AND MEDICATION MANAGEMENT METHOD

(71) Applicant: FREUND CORPORATION, Tokyo (JP)

(72) Inventor: Kiyoshi IMAI, Tokyo (JP)

(21) Appl. No.: 16/297,042

(22) Filed: Mar. 8, 2019

(30) Foreign Application Priority Data
Mar. 22, 2018 (JP) 2018-053850

(51) Int. Cl. A61J 3/00; B41F 17/36; A61J 7/04

(52) U.S. Cl. CPC A61J 3/007; G16H 20/10; A61J 7/04; B41F 17/36

(57) ABSTRACT

A solid pharmaceutical preparation T includes individual identification information 2 that enables identification of an individual who should take the solid pharmaceutical preparation T by visual observation printed on the solid pharmaceutical preparation T, in addition to pharmaceutical preparation information 1 indicated on the solid pharmaceutical preparation T.

### TECHNICAL FIELD

The present invention relates to a solid pharmaceutical preparation and a medication management method that enable an identification of an individual who should take the solid pharmaceutical preparation.

### BACKGROUND ART

In recent years, solid pharmaceutical preparations such as tablets and capsules have the name of the product or the logotype of the company and the like (to be defined as “pharmaceutical preparation information” herein) printed thereon in letters to indicate the kind of the solid pharmaceutical preparation and the manufacturer, which makes it easier to identify the type of the medicine, resulting in a decreasing risk of dispensing errors by pharmacists.

On the other hand, in nursing homes and the like, a plurality of persons may have a meal at the same time and take medicines at the same time. Because of this, recently, there is increased needs for multi-dose packaging, i.e., enclosing a plurality of solid pharmaceutical preparations that each person takes in a sealed pack.

Patent Literature 1 recites that the timing of administration and the name of the patient are printed on each multi-dose pack.

### CITATION LIST

### Patent Literature

Patent Literature 1: Japanese Laid-Open Patent Application Publication No. 2016-126361.

### SUMMARY OF INVENTION

### Technical Problem

However, when a solid pharmaceutical preparation, such as a tablet, taken out of a multi-dose pack is mixed in solid pharmaceutical preparations for another person due to a mistake of a caregiver, a fall of the preparation, or the like, it is no longer possible to identify the solid pharmaceutical preparation for each person, which is problematic.

An object of the present invention is to solve the above-described problem and to enable an identification of an individual who should take each solid pharmaceutical preparation.

### Solution to Problem

To solve the above-described problem, the present invention provides a solid pharmaceutical preparation that enables an identification of an individual who should take the solid pharmaceutical preparation, the solid pharmaceutical preparation including individual identification information that enables the identification of the individual who should take the solid pharmaceutical preparation by visual observation printed on the solid pharmaceutical preparation, in addition to pharmaceutical preparation information indicated on the solid pharmaceutical preparation.

According to this configuration, the individual who should take each solid pharmaceutical preparation can be identified at a glance.

The individual identification information may be a name of the individual who should take the solid pharmaceutical preparation.

According to this configuration, anyone can identify the individual who should take the solid pharmaceutical preparation at a glance.

The individual identification information may be printed in such a way as not to overlap the indication of the pharmaceutical preparation information.

According to this configuration, both the individual identification information and the pharmaceutical preparation information can be identified.

The individual identification information may be printed on a side surface of the solid pharmaceutical preparation.

According to this configuration, both the individual identification information and the pharmaceutical preparation information can be identified since the individual identification information is printed in such a way as not to overlap the indication of the pharmaceutical preparation information on the upper surface or the lower surface of the solid pharmaceutical preparation.

The individual identification information may be printed in a different color from a color in which the pharmaceutical preparation information is indicated.

According to this configuration, the individual identification information and the pharmaceutical preparation information can be clearly distinguished and identified.

Medication timing information may be printed in addition to the individual identification information and the pharmaceutical preparation information.

According to this configuration, failures to take medicine and mistakes of taking a wrong medicine can be prevented.

To solve the above-described problem, the present invention also provides a medication management method for a solid pharmaceutical preparation that enables an identification of an individual who should take the solid pharmaceutical preparation, the method including printing individual identification information that enables the identification of the individual who should take the solid pharmaceutical preparation by visual observation on the solid pharmaceutical preparation at a time of dispensing, in addition to pharmaceutical preparation information indicated on the solid pharmaceutical preparation.

According to this configuration, the individual who should take each solid pharmaceutical preparation can be identified. In particular, by printing individual identification information of the individual who should take the solid pharmaceutical preparation at the time of dispensing, such as in the process of multi-dose packaging, it is possible to prevent mistakes in multi-dose packaging and mistakes of confusing a solid pharmaceutical preparation taken out of a multi-dose package with a solid pharmaceutical preparation for another person.

The individual identification information may be a name of the individual who should take the solid pharmaceutical preparation.

According to this configuration, anyone can identify the individual who should take the solid pharmaceutical preparation at a glance.

The individual identification information may be printed in such a way as not to overlap the indication of the pharmaceutical preparation information.

According to this configuration, both the individual identification information and the pharmaceutical preparation information can be identified.

The individual identification information may be printed on a side surface of the solid pharmaceutical preparation.

According to this configuration, when there is no room as pharmaceutical preparation information is indicated on the upper surface and on the lower surface of the solid pharmaceutical preparation, individual identification information can be printed in such a way as not to overlap the indication of the pharmaceutical preparation information and thereby both the individual identification information and the pharmaceutical preparation information can be identified.

The individual identification information may be printed in a different color from a color in which the pharmaceutical preparation information is indicated.

According to this configuration, the individual identification information and the pharmaceutical preparation information can be clearly distinguished and identified.

Medication timing information may be printed in addition to the individual identification information and the pharmaceutical preparation information.

According to this configuration, failures to take medicine and mistakes of taking a wrong medicine can be prevented.

### Advantageous Effects of Invention

A solid pharmaceutical preparation and a medication management method of the present invention enable an identification of an individual who should take each solid pharmaceutical preparation.

### BRIEF DESCRIPTION OF DRAWINGS

FIG. 1 is a diagram for describing a solid pharmaceutical preparation according to Embodiment 1 of the present invention, FIG. 1 (a) illustrating an example of a tablet with individual identification information printed thereon in addition to an indication of pharmaceutical preparation information and FIG. 1 (b) illustrating an example of a irregularly shaped tablet with individual identification information printed thereon in addition to an indication of pharmaceutical preparation information.

FIG. 2 is a diagram for describing a medication management method according to Embodiment 1 of the present invention.

FIG. 3 is a diagram for describing a solid pharmaceutical preparation according to Embodiment 2 of the present invention, FIG. 3 (a) being a plan view and

FIG. 3 (b) being a side view.

FIG. 4 is a diagram for describing a solid pharmaceutical preparation according to Embodiment 5 of the present invention, FIG. 4 (a) being a plan view and FIG. 4 (b) being a side view.

### DESCRIPTION OF EMBODIMENTS

### Embodiment 1

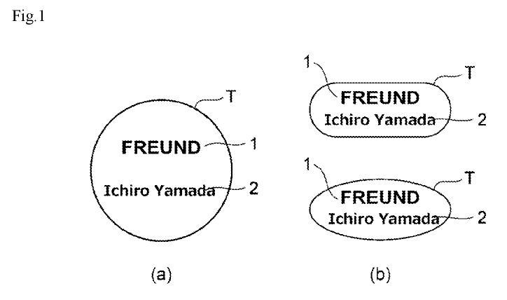

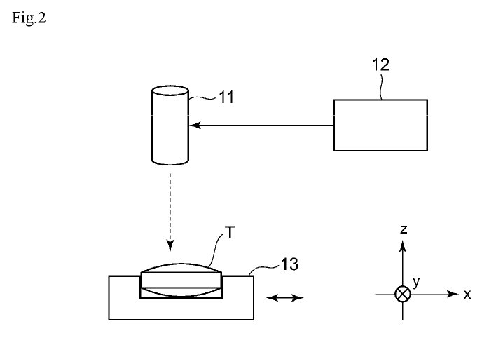

Embodiment 1 of the present invention will be described with reference to FIGS. 1 and 2. FIG. 1 is a diagram for describing a solid pharmaceutical preparation according to Embodiment 1 of the present invention, FIG. 1 (a) illustrating an example of a tablet with individual identification information printed thereon in addition to an indication of pharmaceutical preparation information and FIG. 1 (b) illustrating an example of a irregularly shaped tablet with individual identification information printed thereon in addition to an indication of pharmaceutical preparation information. FIG. 2 is a diagram for describing a medication management method according to Embodiment 1 of the present invention.

### (Solid Pharmaceutical Preparation)

In the present invention, the term “solid pharmaceutical preparation” is defined to include a tablet, an irregularly shaped tablet, a capsule, and other solid pharmaceutical preparations. One of the main surfaces of a solid pharmaceutical preparation is called the upper surface and the other of the main surface is called the lower surface. The surface constituting the circumferential side surface relative to the upper surface and the lower surface is called the side surface.

FIG. 1 (a) illustrates a case in which the solid pharmaceutical preparation T is a tablet and FIG. 1 (b) illustrates a case in which the solid pharmaceutical preparation T is an irregularly shaped tablet. The solid pharmaceutical preparation T has a pharmaceutical preparation information 1 indicated thereon by print or impression by the pharmaceutical company. The pharmaceutical preparation information 1 may be the name of the pharmaceutical company, medicinal contents, the name of the solid pharmaceutical preparation, an identification code, or the like.

An individual identification information 2 is printed beneath the pharmaceutical preparation information 1 as information that enables the identification of the individual who should take the solid pharmaceutical preparation by visual observation in such a way as not to overlap the pharmaceutical preparation information 1. The individual identification information 2 is printed mainly at the time of dispensing involving multi-dose packaging, the detail of which will be described later.

In Embodiment 1, the name of the individual who should take the solid pharmaceutical preparation is printed as the individual identification information 2. For example, the first name and the surname (e.g., “Ichiro Yamada”), the surname alone (e.g., “Yamada”), or the first name alone (e.g., “Ichiro”) is printed in Chinese characters, katakana, hiragana, the Roman alphabet, or other letters.

Note that, although a name that enables the identification of the individual who should take the solid pharmaceutical preparation by visual observation is printed as the individual identification information 2 in Embodiment 1, the configuration is not necessarily limited thereto and may be altered as appropriate. For example, when the individual who should take the solid pharmaceutical preparation usually likes a certain character and a third person can associate the character with the individual upon seeing the character, the character may be printed as the individual identification information 2. Further, the individual identification information 2 may be a family crest, a mark, a geometric figure such as a rectangle, a triangle, or the like, but the present invention does not include information that cannot be identified by visual observation, such as two-dimensional bar codes and barcodes.

In other words, the individual identification information 2 that enables the identification of the individual who should take the solid pharmaceutical preparation by visual observation may be printed.

In the examples in FIGS. 1 (a) and 1 (b), a name is printed as the individual identification information 2. In both cases, a name is printed beneath and alongside the pharmaceutical preparation information 1 as information that enables the identification of the individual who should take the solid pharmaceutical preparation by visual observation in such a way as not to overlap the pharmaceutical preparation information 1.

Note that, although the individual identification information 2 is printed beneath and alongside the pharmaceutical preparation information 1 in Embodiment 1, the configuration is not necessarily limited thereto and may be altered as appropriate. For example, when there are wide spaces between the letters of the pharmaceutical preparation information 1, the individual identification information 2 may be printed in the same row as the pharmaceutical preparation information 1, in the spaces between the letters, or the individual identification information 2 may be printed in a way as to form an angle with the pharmaceutical preparation information 1. The individual identification information 2 may be printed in such a way as to at least enable the identification of the individual by visual observation. Preferably, it is desirable that the individual identification information 2 be printed in such a way as not to overlap the pharmaceutical preparation information 1. This allows both the pharmaceutical preparation information 1 and the individual identification information 2 to be identified.

### (Medication Management Method)

Next, a medication management method will be described with reference to FIG. 2. When solid pharmaceutical preparations T are dispensed to patients at a pharmacy, a hospital dispensary, or the like, a plurality of solid pharmaceutical preparation T to be taken at the same time are often enclosed in a sealed pack (multi-dose packaging). This prevents mistakes of taking a wrong medicine.

On the other hand, in nursing homes and the like, a plurality of persons may have a meal at the same time and take medicines at the same time. In such an occasion, when a solid pharmaceutical preparation, such as a tablet, taken out of a pack of multi-dose packaging is mixed in solid pharmaceutical preparations for other persons due to a mistake of a caregiver, a fall of the preparation, or the like, it is no longer possible to identify the solid pharmaceutical preparation for each person, which is problematic.

To address this problem, according to Embodiment 1 of the present invention, the individual identification information 2 that enables the identification of the individual who should take the solid pharmaceutical preparation by visual observation is printed on the solid pharmaceutical preparations T before the solid pharmaceutical preparations are enclosed in sealed packs in the process of multi-dose packaging at the time of dispensing at a pharmacy, a hospital dispensary, or the like. In Embodiment 1, a name is printed as the individual identification information 2 that enables the identification of the individual by visual observation.

Note that, although a name that enables the identification of the individual who should take the solid pharmaceutical preparation by visual observation is printed as the individual identification information 2 in Embodiment 1, the configuration is not necessarily limited thereto and may be altered as appropriate. For example, when the individual who should take the solid pharmaceutical preparation usually likes a certain character and a third person can associate the character with the individual upon seeing the character, the character may be printed as the individual identification information 2. Similarly, a family crest, a mark, a geometric figure such as a rectangle, a triangle, or the like may be printed as the individual identification information 2, but the present invention does not include information that cannot be identified by visual observation, such as two-dimensional bar codes and barcodes. In other words, the individual identification information 2 that enables the identification of the individual who should take the solid pharmaceutical preparation by visual observation may be printed.

Note that, although the individual identification information 2 is printed on the solid pharmaceutical preparations T at the time of dispensing involving the process of multi-dose packaging in Embodiment 1, the configuration is not necessarily limited thereto and may be altered as appropriate. For example, the individual identification information 2 may be printed on the solid pharmaceutical preparation T at the time of dispensing not involving the process of multi-dose packaging.

Printing is performed on each solid pharmaceutical preparation T with edible ink discharged by an inkjet head 11. The inkjet head 11 is equipped with a plurality of nozzles in the direction away from the viewer in FIG. 2 (in the y-direction) and can discharge edible ink in a row.

The solid pharmaceutical preparation T is placed on a holder 13, the lower surface of the solid pharmaceutical preparation facing downward. The holder 13 is movable in the x-direction and, by discharging edible ink from the inkjet head 11 in the y-direction in a row and moving the holder 13 synchronously in the x-direction, the individual identification information 2 can be printed on the upper surface of the solid pharmaceutical preparation T in two dimensions. The movement of the holder 13 and the discharge of edible ink from the nozzles of the inkjet head 11 can be synchronously controlled by using the control unit 12.

The individual identification information 2 to be printed is preferably positioned and printed in such a way as not to overlap the pharmaceutical preparation information 1 already indicated. The printing position is designated by using the control unit 12 in order to do so. The control unit 12 includes a personal computer and is configured to allow the designation of a printing position with a view on the screen. In this process, an image of the upper surface of the solid pharmaceutical preparation T placed on the holder 13 is captured by a camera (not illustrated) and the position and angle at which to print the individual identification information 2 is designated on the screen in such a way as not to overlap the pharmaceutical preparation information 1.

Alternatively, the position and angle at which to print the individual identification information 2 may be automatically determined by processing the image captured by the camera for automatic recognition of the position at which the pharmaceutical preparation information 1 is indicated.

Note that, although the position and angle of the individual identification information 2 is designated in such a way as not to overlap the pharmaceutical preparation information 1 in Embodiment 1, the configuration is not necessarily limited thereto and may be altered as appropriate. For example, a position and angle may be designated in such a way that the individual who should take the solid pharmaceutical preparation is identifiable by visual observation even though the pharmaceutical preparation information 1 and the individual identification information 2 overlap each other to some extent.

Further, although the control unit 12 is configured by a personal computer in Embodiment 1, the configuration is not necessarily limited thereto and may be altered as appropriate. For example, the control unit 12 may be configured by a combination of a control circuit and a display device other than a personal computer and may be configured by a mobile terminal such as a smart phone.

Further, although edible ink is discharged from the inkjet head 11 in the y-direction in a row and the holder 13 is synchronously moved in the x-direction in Embodiment 1, the configuration is not necessarily limited thereto and may be altered as appropriate. For example, edible ink may be discharged from the inkjet head 11 in the x-direction in a row with the holder 13 synchronously moved in the y-direction or edible ink may be discharged from the inkjet head 11 in the y-direction in a row with the inkjet head 11 moved in the x-direction.

Further, although the printing is applied on the solid pharmaceutical preparation T by discharging edible ink from the inkjet head 11 in Embodiment 1, the configuration is not necessarily limited thereto and may be altered as appropriate. For example, edible ink may be applied to the solid pharmaceutical preparation T by screen printing or may be applied to the solid pharmaceutical preparation T by any technique.

Thus, the individual who should take each solid pharmaceutical preparation can be identified in Embodiment 1, owing to a solid pharmaceutical preparation that enables an identification of an individual who should take the solid pharmaceutical preparation, the solid pharmaceutical preparation including individual identification information that enables the identification of the individual who should take the solid pharmaceutical preparation by visual observation printed on the solid pharmaceutical preparation, in addition to pharmaceutical preparation information indicated on the solid pharmaceutical preparation.

Further, the individual who should take each solid pharmaceutical preparation can be identified, owing to a medication management method for a solid pharmaceutical preparation that enables an identification of an individual who should take the solid pharmaceutical preparation, the method including printing individual identification information that enables the identification of the individual who should take the solid pharmaceutical preparation by visual observation on the solid pharmaceutical preparation at a time of dispensing, in addition to pharmaceutical preparation information indicated on the solid pharmaceutical preparation.

### Embodiment 2

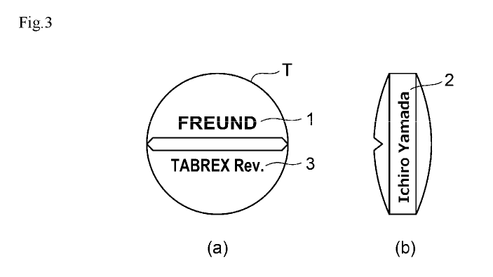

Embodiment 2 of the present invention differs from Embodiment 1 in that the individual identification information is printed on the side surface of the solid pharmaceutical preparation. Embodiment 2 will be described with reference to FIG. 3. FIG. 3 is a diagram for describing a solid pharmaceutical preparation according to Embodiment 2 of the present invention, FIG. 3 (a) being a plan view and FIG. 3 (b) being a side view.

On the upper surface of a solid pharmaceutical preparation T according to Embodiment 2, another piece of pharmaceutical preparation information 3 is indicated by the pharmaceutical company in addition to the pharmaceutical preparation information 1 as illustrated in FIG. 3(a). Further, as illustrated in FIG. 3(b), the name of the individual who should take the solid pharmaceutical preparation is printed on the side surface of the solid pharmaceutical preparation T as the individual identification information 2. This is because there is not much space on the upper surface of the solid pharmaceutical preparation T in which to print the individual identification information 2 since there is a splitting line between the pharmaceutical preparation information 1 and the pharmaceutical preparation information 3 on the upper surface.

Thus, the pharmaceutical preparation information 1 and the pharmaceutical preparation information 3 can be identified and the individual who should take the solid pharmaceutical preparation can be identified by visual observation owing to the individual identification information 2 since the pharmaceutical preparation information 1 and the pharmaceutical preparation information 3 are indicated on the upper surface of the solid pharmaceutical preparation T and the individual identification information 2, which is a name, that enables the identification of the individual who should take the solid pharmaceutical preparation by visual observation is printed on the side surface in such a way as not to overlap the pharmaceutical preparation information 1 or the pharmaceutical preparation information 3. Further, since the name is printed as the individual identification information 2, anyone can identify the individual who should take the solid pharmaceutical preparation at a glance.

Further, although a name is printed as the individual identification information 2 also in Embodiment 2, the configuration is not necessarily limited thereto and may be altered as appropriate. For example, a character that the individual usually likes and that enables the identification of the individual, a family crest, a mark, or a geometric figure may be printed.

In the case in which the individual identification information 2 is to be printed on the side surface of the solid pharmaceutical preparation T, the solid pharmaceutical preparation T is placed on the holder 13 to stand in the z-direction, with the side surface facing the inkjet head 11, and edible ink is discharged from the nozzles of the inkjet head 11 arranged in the y-direction as in Embodiment 1, and the holder 13 is synchronously moved in the x-direction to print the individual identification information 2, thus constituting the medication management method according to Embodiment 2.

Alternatively, the solid pharmaceutical preparation T may be placed on the holder 13 to stand with the upper surface and the lower surface facing the x-direction and the side surface facing the inkjet head 11 (i.e., in the z-direction), edible ink discharged respectively from the nozzles of the inkjet head 11 arranged in the x-direction and the solid pharmaceutical preparation T rotated synchronously with the rotation axis kept in position.

Thus, in Embodiment 2, printing the individual identification information 2 on the side surface of the solid pharmaceutical preparation T allows the individual identification information not to overlap the pharmaceutical preparation information indicated on the upper surface or the lower surface of the solid pharmaceutical preparation and thereby enables both the individual identification information and the pharmaceutical preparation information to be identified.

### Embodiment 3

Embodiment 3 of the present invention differs from Embodiment 1 in that the individual identification information is printed on the lower surface of the solid pharmaceutical preparation.

In other words, in Embodiment 3, the name of the individual who should take the solid pharmaceutical preparation is printed on the lower surface of the solid pharmaceutical preparation T as the individual identification information 2. This is because there is a splitting line (not illustrated) between the pharmaceutical preparation information 1 and the pharmaceutical preparation information 3 on the upper surface of the solid pharmaceutical preparation T and there is not much space on the upper surface in which to print the individual identification information 2. The pharmaceutical preparation information 1 and the pharmaceutical preparation information 3 as well as the individual identification information 2 can be identified also in Embodiment 3. Further, since the name is printed as the individual identification information 2, anyone can identify the individual who should take the solid pharmaceutical preparation at a glance.

To print the individual identification information 2 on the lower surface of the solid pharmaceutical preparation T, the solid pharmaceutical preparation T is placed on the holder 13 with the lower surface facing the inkjet head 11 and, as in Embodiments 1 and 2, edible ink is discharged from the nozzles of the inkjet head 11 and the holder 13 is synchronously moved in the x-direction to print the individual identification information 2, thus constituting the medication management method according to Embodiment 3.

Thus, the pharmaceutical preparation information 1 and the pharmaceutical preparation information 3 can be identified and the individual who should take the solid pharmaceutical preparation can be identified by visual observation owing to the individual identification information 2 since the pharmaceutical preparation information 1 and the pharmaceutical preparation information 3 are indicated on the upper surface of the solid pharmaceutical preparation T and the name is printed on the lower surface as the individual identification information 2 that enables the identification of the individual who should take the solid pharmaceutical preparation by visual observation in such a way as not to overlap the pharmaceutical preparation information 1 or the pharmaceutical preparation information 3.

Further, although a name is printed as the individual identification information 2 also in Embodiment 3, the configuration is not necessarily limited thereto and may be altered as appropriate. For example, a character that the individual usually likes and that enables the identification of the individual, a family crest, a mark, or a geometric figure may be printed.

Thus, in Embodiment 3, printing the individual identification information 2 on the lower surface of the solid pharmaceutical preparation T allows the individual identification information to be printed in such a way as not to overlap the indication of the pharmaceutical preparation information printed on the upper surface or the side surface of the solid pharmaceutical preparation and thereby enables both the individual identification information and the pharmaceutical preparation information to be identified.

### Embodiment 4

Embodiment 4 of the present invention differs from Embodiments 1 to 3 in that the individual identification information is printed in a different color from a color in which the pharmaceutical preparation information is indicated.

The surface of the solid pharmaceutical preparation T according to Embodiment 4 is white and the pharmaceutical preparation information 1 is printed and indicated in black. The individual identification information 2 is printed in red, a different color from the black color in which the pharmaceutical preparation information 1 is printed. This enables both the individual identification information 2 and the pharmaceutical preparation information 1 to be clearly distinguished and identified even when the individual identification information 2 and the pharmaceutical preparation information 1 overlap each other to some extent.

In a medication management method according to Embodiment 4, the inkjet head 11 may be filled with red edible ink to perform printing. Alternatively, an inkjet head 11 filled with black edible ink may be provided alongside the inkjet head 11 filled with black edible ink and an instruction to discharge red edible ink may be given by using the control unit 12.

Note that, although the individual identification information 2 in Embodiment 4 is printed in red to be in a different color from a color in which the pharmaceutical preparation information 1 is indicated, the configuration is not necessarily limited thereto and may be altered as appropriate. For example, the individual identification information 2 may be printed in any color, for example, blue or yellow.

Further, although the surface of the solid pharmaceutical preparation T is white in Embodiment 4, the configuration is not necessarily limited thereto and may be altered as appropriate. For example, the surface of the solid pharmaceutical preparation T may be orange or may be light blue.

Thus, in Embodiment 4, the individual identification information and the pharmaceutical preparation information can be clearly distinguished and identified since the individual identification information is printed in a different color from a color in which the pharmaceutical preparation information is indicated.

### Embodiment 5

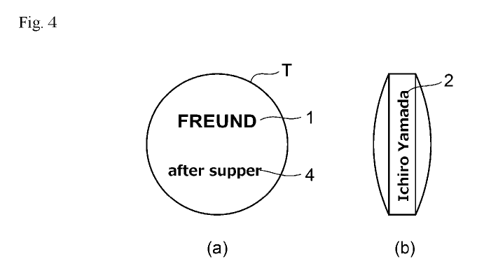

Embodiment 5 of the present invention differs from Embodiments 1 to 4 in that medication timing information is printed on the solid pharmaceutical preparation, in addition to the printing of the individual identification information and the indication of pharmaceutical preparation information. Embodiment 5 will be described with reference to FIG. 4. FIG. 4 is a diagram for describing a solid pharmaceutical preparation according to Embodiment 5 of the present invention, FIG. 4(a) being a plan view and FIG. 4(b) being a side view.

As illustrated in FIG. 4(a), pharmaceutical preparation information 1 is indicated and a medication timing information 4 “after supper” is printed alongside the pharmaceutical preparation information 1 on the upper surface of the solid pharmaceutical preparation T. Further, as illustrated in FIG. 4(b), the individual identification information 2 is printed on the side surface of the solid pharmaceutical preparation T. This allows the medication timing information 4 in addition to the pharmaceutical preparation information 1 and the individual identification information 2 to be visually recognized and prevents failures to take medicine and mistakes of taking a wrong medicine.

Note that, although the pharmaceutical preparation information 1 is indicated and the medication timing information 4 is printed on the upper surface of the solid pharmaceutical preparation T and the individual identification information 2 is printed on the side surface of the solid pharmaceutical preparation T in Embodiment 5, the configuration is not necessarily limited thereto and may be altered as appropriate. For example, the upper surface of the solid pharmaceutical preparation T may have pharmaceutical preparation information 1 indicated thereon and the individual identification information 2 printed thereon, the side surface of the solid pharmaceutical preparation T having the medication timing information 4 printed thereon, or the upper surface of the solid pharmaceutical preparation T may have pharmaceutical preparation information 1 indicated thereon, the lower surface of the solid pharmaceutical preparation T having the individual identification information 2 printed thereon, the side surface of the solid pharmaceutical preparation T having the medication timing information 4 printed thereon. In other words, the medication timing information 4 may be printed on any of the surfaces, in addition to the printing of individual identification information and the indication of pharmaceutical preparation information.

Further, apart from “after supper”, the medication timing information 4 may be “after breakfast” or “after lunch”, or simply “morning”, “afternoon”, or “evening”. Further, the time of medication, for example, “12 o'clock” or “6:30” may be printed.

Further, the date of medication may be printed, for example, as “the Xth day of the month of Y” or the time and date may be printed, for example, as “Z hours on the Xth day of the month of Y”. In other words, the timing for medication may be printed in any appropriate expression.

Thus, in Embodiment 5, printing medication timing information on the solid pharmaceutical preparation, in addition to the printing of individual identification information and the indication of pharmaceutical preparation information, prevents failures to take medicine and mistakes of taking a wrong medicine.

### INDUSTRIAL APPLICABILITY

A solid pharmaceutical preparation and a medication management method of the present invention can be widely applied to areas of medication management of solid pharmaceutical preparations.

### REFERENCE SIGNS LIST

1 pharmaceutical preparation information
2 individual identification information
3 pharmaceutical preparation information
4 medication timing information
T solid pharmaceutical preparation
11 inkjet head
12 control unit
13 holder

1. A solid pharmaceutical preparation that enables an identification of an individual who should take the solid pharmaceutical preparation, the solid pharmaceutical preparation comprising individual identification information that enables the identification of the individual who should take the solid pharmaceutical preparation by visual observation printed on the solid pharmaceutical preparation, in addition to pharmaceutical preparation information indicated on the solid pharmaceutical preparation.

2. The solid pharmaceutical preparation according to claim 1, wherein the individual identification information is a name of the individual who should take the solid pharmaceutical preparation.

3. The solid pharmaceutical preparation according to claim 1, wherein the individual identification information is printed in such a way as not to overlap the indication of the pharmaceutical preparation information.

4. The solid pharmaceutical preparation according to claim 1, wherein the individual identification information is printed on a side surface of the solid pharmaceutical preparation.

5. The solid pharmaceutical preparation according to claim 1, wherein the individual identification information is printed in a different color from a color in which the pharmaceutical preparation information is indicated.

6. The solid pharmaceutical preparation according to claim 1, wherein medication timing information is printed in addition to the individual identification information and the pharmaceutical preparation information.

7. A medication management method for a solid pharmaceutical preparation that enables an identification of an individual who should take the solid pharmaceutical preparation, the method comprising printing individual identification information that enables the identification of the individual who should take the solid pharmaceutical preparation by visual observation on the solid pharmaceutical preparation at a time of dispensing, in addition to pharmaceutical preparation information indicated on the solid pharmaceutical preparation.

8. The medication management method according to claim 7, wherein the individual identification information is a name of the individual who should take the solid pharmaceutical preparation.

9. The medication management method according to claim 7, wherein the individual identification information is printed in such a way as not to overlap the indication of the pharmaceutical preparation information.

10. The medication management method according to claim 7, wherein the individual identification information is printed on a side surface of the solid pharmaceutical preparation.

11. The medication management method according to claim 7, wherein the individual identification information is printed in a different color from a color in which the pharmaceutical preparation information is indicated.

12. The medication management method according to claim 7, wherein medication timing information is printed in addition to the individual identification information and the pharmaceutical preparation information.
